(19) United States
(12) Patent Application Publication
(10) Pub. No.: US 2011/0274903 A1
(43) Pub. Date: Nov. 10, 2011

(54) WEIGHTED FABRIC ARTICLES AND RELATED MATERIALS AND METHODS

(76) Inventors: Charlene Stuart, Northfield, OH (US); Patricia Dorton, Cuyahoga Falls, OH (US)

(21) Appl. No.: 13/103,055

(22) Filed: May 7, 2011

Related U.S. Application Data
(60) Provisional application No. 61/332,252, filed on May 7, 2010.

(51) Int. Cl. D02G 3/44; B32B 5/02; D02G 3/36; A41D 13/00; A41D 1/00; A41D 1/02; A41D 1/04; A41B 1/00; A41D 1/06; A41D 1/14; C08F 20/12; C08F 16/06; C08F 14/06; C08F 20/44; C08F 10/00; C08F 10/06; C08F 10/02; C08G 18/32; C08G 8/10; C08G 63/183; C08G 69/26; C08G 69/12; C08G 63/00; C08L 29/04; C08L 33/08; C08L 33/20; C08L 27/06; C08L 23/12; C08L 23/06; C08L 75/12; C08L 61/06; C08L 67/02; C08L 77/06; C08L 67/00; C08K 3/00; C08K 3/22; C08L 77/10; C08K 3/34; C08B 16/00; C03C 13/00; C03C 14/00; C07C 31/18; C09D 101/02; D02G 3/04

(52) U.S. Cl. 428/219; 428/373; 428/221; 442/181; 2/455; 2/69; 2/93; 2/102; 526/328; 525/56; 526/344.3; 526/341; 526/342; 526/348; 526/351; 526/352; 528/44; 528/165; 528/308.1; 528/308.6; 528/335; 528/331; 528/271; 524/557; 524/560; 524/566; 524/567; 524/584; 524/586; 524/590; 524/594; 524/605; 524/606; 524/599; 524/424; 524/430; 536/57; 501/35; 501/32; 568/852; 106/163.01

(57) ABSTRACT

Embodiments of the invention may include one or more high-density textile fibers with a density from about 1 to about 11 g/mL. Some embodiments may include fabrics and/or garments made from such fibers. Embodiments comprising garments can include, for instance, exercise aids for providing extra mass to the wearer. Other embodiments can comprise, without limitation, building materials, safety equipment, armor, body armor, protective apparel, marine repair materials, marine protective devices, boat hulls, vehicle chassis, or blast-resistant materials.

This application claims priority to U.S. Provisional Patent Application Ser. No. 61/332,252 entitled Weighted Fabric Articles and Related Materials and Methods filed on May 7, 2010 and now pending, which is hereby incorporated by reference in its entirety.

### I. BACKGROUND OF THE INVENTION

A. Field of Invention

The present invention generally relates to high-density textile fibers, yarns, fabrics, and articles made therefrom, as well as related materials and methods.

B. Description of the Related Art

It is known to use weighted garments for exercise applications. However, existing weighted garments have a variety of shortcomings. For instance, many are made from ordinary textile fibers adapted from non-weighted applications. Weight is added to such garments by adding dense non-fabric materials. For example, such dense materials may be sewn into the garment in patches or layers, or they may be incorporated into a pocket or pouch within the garment. This results in a garment that is difficult to wear due to shifting and/or unbalanced loading. Furthermore, such a garment is typically bulky and uncomfortable to wear.

Weighted fibers are known in the textile arts. For instance, it is known to have a conjugate fiber structure where the sheath is made from polyethylene or polyester and the core is made from a lead or tungsten oxide impregnated resin. Further, some known conjugate fibers have a lead alloy core, and a sheath made from a polyester. Other weighted fibers are also know such as stainless steel, copper, aluminum, nylon, Kevlar®, Spectra®, nickel, glass or carbon. However, these and other known weighted fibers are not suitable for weighted textile fabrics because they lack, for example, sufficient density, elasticity, and/or flexibility.

What is needed is a textile fiber having mechanical properties compatible with forming high-density garments therefrom, and sufficient density to provide a physiological benefit through exercise. Some embodiments of the present invention may provide one or more of these benefits and/or may overcome one or more deficiencies of the prior art.

### II. SUMMARY OF THE INVENTION

Some embodiments may relate to a textile fiber, comprising a density from about 1 to about 11 g/mL.

Some embodiments may further comprise one or more of a glass fiber, a metal fiber, a conjugate fiber, a high-density polymer fiber, or a filled fiber.

In some embodiments the fiber further comprises a matrix material selected from one or more of polyacetate, polyacrylate, polyacrylic, lyocell, modacrylic, nylon, polyamide nylon, polyphenol formaldehyde, polyvinyl alcohol, polyvinyl chloride, polyester, PET polyester, PBT polyester, acrylic polyesters, polyacrylonitrile, polyaramids, polyolefin, polypropylene, polyethylene, high performance polyethylene, ultra-high molecular weight polyethylene, polyurethane, polyurethane-polyurea copolymer, poly-1,3-propanediol, fiber glass, E-fiberglass, A-fiberglass, E-CR-fiberglass, C-fiberglass, D-fiberglass, R-fiberglass, or S-fiberglass, or any blend or copolymer thereof.

In some embodiments the matrix material contains a filler material selected from one or more of metals, metal oxides, carbides, ceramics; or silicates.

In some embodiments wherein the fiber further comprises: a core material selected from one or more of polyacetate, polyacrylate, polyacrylic, lyocell, modacrylic, nylon, polyamide nylon, polyphenol formaldehyde, polyvinyl alcohol, polyvinyl chloride, polyester, PET polyester, PBT polyester, acrylic polyesters, polyacrylonitrile, polyaramids, polyolefin, polypropylene, polyethylene, high performance polyethylene, ultra-high molecular weight polyethylene, polyurethane, polyurethane-polyurea copolymer, poly-1,3-propanediol, fiber glass, E-fiberglass, A-fiberglass, E-CR-fiberglass, C-fiberglass, D-fiberglass, R-fiberglass, or S-fiberglass, metals, copper, gold, silver, nickel, aluminum, iron, ferrous alloys, steel, spring steel, or stainless steel; and a sheath material selected from one or more of polyacetate, polyacrylate, polyacrylic, lyocell, modacrylic, nylon, polyamide nylon, polyphenol formaldehyde, polyvinyl alcohol, polyvinyl chloride, polyester, PET polyester, PBT polyester, acrylic polyesters, polyacrylonitrile, polyaramids, polyolefin, polypropylene, polyethylene, high performance polyethylene, ultra-high molecular weight polyethylene, polyurethane, polyurethane-polyurea copolymer, poly-1,3-propanediol, fiber glass, E-fiberglass, A-fiberglass, E-CR-fiberglass, C-fiberglass, D-fiberglass, R-fiberglass, or S-fiberglass, metals, copper, gold, silver, nickel, aluminum, iron, ferrous alloys, steel, spring steel, stainless steel, or any blend or copolymer thereof.

Some embodiments may relate to a textile yarn comprising at least a high-density textile fiber having a density from about 1 to about 11 g/mL.

In some embodiments the high-density textile fiber comprises one or more of a glass fiber, a metal fiber, a conjugate fiber, a high-density polymer fiber, or a filled fiber.

In some embodiments the filled fiber further comprises a matrix material selected from one or more of polyacetate, polyacrylate, polyacrylic, lyocell, modacrylic, nylon, polyamide nylon, polyphenol formaldehyde, polyvinyl alcohol, polyvinyl chloride, polyester, PET polyester, PBT polyester, acrylic polyesters, polyacrylonitrile, polyaramids, polyolefin, polypropylene, polyethylene, high performance polyethylene, ultra-high molecular weight polyethylene, polyurethane, polyurethane-polyurea copolymer, poly-1,3-propanediol, fiber glass, E-fiberglass, A-fiberglass, E-CR-fiberglass, C-fiberglass, D-fiberglass, R-fiberglass, or S-fiberglass, or any blend or copolymer thereof.

In some embodiments the matrix material of the filled fiber contains a filler material selected from one or more of metals, metal oxides, carbides, ceramics, or silicates.

In some embodiments the conjugate fiber further comprises: a core material selected from one or more of polyacetate, polyacrylate, polyacrylic, lyocell, modacrylic, nylon, polyamide nylon, polyphenol formaldehyde, polyvinyl alcohol, polyvinyl chloride, polyester, PET polyester, PBT polyester, acrylic polyesters, polyacrylonitrile, polyaramids, polyolefin, polypropylene, polyethylene, high performance polyethylene, ultra-high molecular weight polyethylene, polyurethane, polyurethane-polyurea copolymer, poly-1,3-propanediol, fiber glass, E-fiberglass, A-fiberglass, E-CR-fiberglass, C-fiberglass, D-fiberglass, R-fiberglass, or S-fiberglass, metals, copper, gold, silver, nickel, aluminum, iron, ferrous alloys, steel, spring steel, or stainless steel, or any blend or copolymer thereof; and a sheath material selected from one or more of polyacetate, polyacrylate, polyacrylic, lyocell, modacrylic, nylon, polyamide nylon, polyphenol formaldehyde, polyvinyl alcohol, polyvinyl chloride, polyester, PET polyester, PBT polyester, acrylic polyesters, polyacrylonitrile, polyaramids, polyolefin, polypropylene, polyethylene, high performance polyethylene, ultra-high molecular weight polyethylene, polyurethane, polyurethane-polyurea copolymer, poly-1,3-propanediol, fiber glass, E-fiberglass, A-fiberglass, E-CR-fiberglass, C-fiberglass, D-fiberglass, R-fiberglass, or S-fiberglass, metals, copper, gold, silver, nickel, aluminum, iron, ferrous alloys, steel, spring steel, or stainless steel, or any blend or copolymer thereof.

Some embodiments further comprise a textile fiber selected from one or more conventional textile fibers.

Some embodiments may generally relate to a textile fabric comprising one or more of a first yarn including a high-density fiber having a density from about 1 to about 11 g/mL.

In some embodiments wherein the first yarn comprises one or more of a glass fiber, a metal fiber, a conjugate fiber, a high density polymer fiber, or a filled fiber.

Some embodiments may further comprise a reinforcing member adapted to support the weight of the fabric and mitigate or prevent the fabric from sagging under its own weight.

In some embodiments the fabric weighs from about 1 lb/yd² to about 20 lb/yd².

Some embodiments may relate to an article including a textile fiber, comprising a density from about 1 to about 11 g/mL.

Some embodiments further comprise a yarn comprising at least a high-density textile fiber having a density from about 1 to about 11 g/mL.

Some embodiments further comprise a fabric comprising one or more of a first yarn including a high-density fiber having a density from about 1 to about 11 g/mL.

In some embodiments the article is selected from one or more of garments, building materials, safety equipment, armor, body armor, protective apparel, marine repair materials, marine protective devices, boat hulls, vehicle chassis, sporting goods, or blast-resistant materials.

In some embodiments a garment can be selected from one or more of shorts, compression shorts, running shorts, a shirt, a jacket, a vest, a harness, pants, a tank top, a skirt, or Capri pants.

### III. BRIEF DESCRIPTION OF THE DRAWINGS

The invention may take physical form in certain parts and arrangement of parts, embodiments of which will be described in detail in this specification and illustrated in the accompanying drawings which form a part hereof and wherein:

FIG. 1 is a perspective view of an embodiment comprising a zip-up jacket;

FIG. 2 is a perspective view of an embodiment comprising a pair of pants;

FIG. 3 is a perspective view of an embodiment comprising a shirt;

FIG. 4 is a perspective view of an embodiment comprising a pair of shorts;

FIG. 5 is a perspective view of an embodiment comprising a vest;

FIG. 6 is a pair of views from opposing sides of an embodiment comprising a singlet;

FIG. 7 is a perspective view of an embodiment comprising a miniskirt;

FIG. 8 is a perspective view of an embodiment comprising a t-shirt;

FIG. 9 is a perspective view of an embodiment comprising a vest;

FIG. 10 is a perspective view of an embodiment comprising a pair of gloves;

FIG. 11 is a perspective view of an embodiment comprising a baseball mitt;

FIG. 12A is a view of an embodiment comprising a football;

FIG. 12B is a view of an embodiment comprising a baseball;

FIG. 12C is a view of an embodiment comprising a soccer ball;

FIG. 13 is a view of an embodiment comprising a golf ball;

FIG. 14 is a perspective view of an embodiment comprising a boat hull;

FIG. 15 is a perspective view of an embodiment comprising a pair of running shoes;

FIG. 16 is a perspective view of an embodiment comprising a pair of shoes;

FIG. 17 is a perspective view of an embodiment comprising a helmet; and

FIG. 18 is a perspective view of an embodiment comprising a body armor vest.

### IV. DETAILED DESCRIPTION OF THE INVENTION

Fibers

The present invention generally relates to high-density textile fibers, and yarns, fabrics, and garments comprising such fibers, and related methods. According to some embodiments, a high density textile fiber can comprise one or more of polyacetate, polyacrylate, polyacrylic, lyocell, modacrylic, nylon, polyamide nylon, polyphenol formaldehyde, polyvinyl alcohol, polyvinyl chloride, polyester, PET polyester, PBT polyester, acrylic polyesters, polyacrylonitrile, polyaramids, polyolefin, polypropylene, polyethylene, high performance polyethylene, ultra-high molecular weight polyethylene, polyurethane, polyurethane-polyurea copolymer, or poly-1,3-propanediol; fiber glass such as, without limitation, one or more of E-glass (alumino-borosilicate glass with less than 1 wt % alkali oxides), A-glass (alkali-lime glass with little or no boron oxide), E-CR-glass (alumino-lime silicate with less than 1 wt % alkali oxides), C-glass (alkali-lime glass with high boron oxide content), D-glass (borosilicate glass with high dielectric constant), R-glass (alumino silicate glass without MgO and CaO with high mechanical requirements), and S-glass (alumino silicate glass without CaO but with high MgO content with high tensile strength); filler materials such as, without limitation, metals, metal oxides, carbides, and/or ceramics; and silicates; metals such as, without limitation, copper, gold, silver, nickel, aluminum, iron, or any alloy thereof including, without limitation, ferrous alloys such as steel, spring steel, or stainless steel. As used herein, the term high-density textile fiber includes fibers having a density greater than 1 g/mL and/or including one or more high-density components such as, without limitation metals, metal oxides, ceramics, carbides, or silicate glasses.

According to some embodiments, a suitable fiber can comprise one or more of a circular cross-section, hollow cross-section, crescent cross-section, oval cross-section, star-shaped cross-section, bilobal cross-section, trilobal cross section, and/or multilobal cross-section.

According to some embodiments, a suitable fiber can comprise a filled fiber having a matrix material, and a filler material dispersed in the matrix material. Suitable filler materials comprise, for instance, high-density particulate materials such as, without limitation, metals, metal oxides, carbides, and/or ceramics; and silicates such as, without limitation aluminosilicates, and/or borosilicates. Suitable matrix materials can include one or more of polyacetate, polyacrylate, polyacrylic, lyocell, modacrylic, nylon, polyamide nylon, polyphenol formaldehyde, polyvinyl alcohol, polyvinyl chloride, polyester, PET polyester, PBT polyester, acrylic polyesters, polyacrylonitrile, polyaramids, polyolefin, polypropylene, polyethylene, high performance polyethylene, ultra-high molecular weight polyethylene, polyurethane, polyurethane-polyurea copolymer, poly-1,3-propanediol, or fiber glass such as, without limitation, one or more of E-glass (alumino-borosilicate glass with less than 1 wt % alkali oxides), A-glass (alkali-lime glass with little or no boron oxide), E-CR-glass (alumino-lime silicate with less than 1 wt % alkali oxides), C-glass (alkali-lime glass with high boron oxide content), D-glass (borosilicate glass with high dielectric constant), R-glass (alumino silicate glass without MgO and CaO with high mechanical requirements), and S-glass (alumino silicate glass without CaO but with high MgO content with high tensile strength).

In some embodiments, a suitable fiber may comprise a core-sheath, conjugate structure, hollow tube structure, or other advantageous forms. According to such embodiments the sheath and/or core may be selected from one or more of polyacetate, polyacrylate, polyacrylic, lyocell, modacrylic, nylon, polyamide nylon, polyphenol formaldehyde, polyvinyl alcohol, polyvinyl chloride, polyester, PET polyester, PBT polyester, acrylic polyesters, polyacrylonitrile, polyaramids, polyolefin, polypropylene, polyethylene, high performance polyethylene, ultra-high molecular weight polyethylene, polyurethane, polyurethane-polyurea copolymer, poly-1,3-propanediol; high-density particulate materials such as, without limitation, metals, metal oxides, carbides, ceramics, silicates; or fiber glass such as, without limitation, one or more of E-glass (alumino-borosilicate glass with less than 1 wt % alkali oxides), A-glass (alkali-lime glass with little or no boron oxide), E-CR-glass (alumino-lime silicate with less than 1 wt % alkali oxides), C-glass (alkali-lime glass with high boron oxide content), D-glass (borosilicate glass with high dielectric constant), R-glass (alumino silicate glass without MgO and CaO with high mechanical requirements), and S-glass (alumino silicate glass without CaO but with high MgO content with high tensile strength), or metals such as, without limitation, copper, gold, silver, nickel, aluminum, iron, or any alloy thereof including, without limitation, ferrous alloys such as steel, spring steel, or stainless steel.

Yarns

According to some embodiments a suitable yarn can comprise at least one kind of high-density fiber, or can comprise a blend of two or more kinds of high-density fibers. In some embodiments a high-density fiber or blend of high-density fibers can be combined with a textile fiber known in the art, including natural and/or synthetic fibers. Thus the density and/or other properties of a yarn can be tuned according to the type of fibers, and proportion of fibers, incorporated therein.

Fabrics

Suitable fabrics comprise high-density fibers as set forth herein, which are incorporated into the fabrics in yarns also as set forth herein. However, suitable fabrics may also include yarns that do not comprise high-density fibers. Thus, a single fabric may comprise one or more of high-density fiber yarns, blended high-density fiber yarns, yarns having a blend of one or more high-density fibers and one or more conventional fibers, or conventional fiber yarns.

According to some embodiments, suitable fabrics can weigh from about 1 lb/yd² to about 20 lb/yd². Other suitable ranges can include from about 1 to 2 lb/yd², from about 2 to 3 lb/yd², from about 3 to 4 lb/yd², from about 4 to 5 lb/yd², from about 5 to 6 lb/yd², from about 6 to 7 lb/yd², from about 7 to 8 lb/yd², from about 8 to 9 lb/yd², from about 9 to 10 lb/yd², from about 10 to 11 lb/yd², from about 11 to 12 lb/yd², from about 12 to 13 lb/yd², from about 13 to 14 lb/yd², from about 14 to 15 lb/yd², from about 15 to 16 lb/yd², from about 16 to 17 lb/yd², from about 17 to 18 lb/yd², from about 18 to 19 lb/yd², or even from about 19 to 20 lb/yd². Here as elsewhere in the specification and claims, ranges may be combined.

According to some embodiments, the yarn or yarns are woven into a fabric using weaving methods for building support structure(s) into the fabric. For example, some embodiments may use, without limitation, Santoni weaving machines and methods for creating such structures. In some embodiments the support structure(s) support at least a portion of the weight of the fabric, and may thereby prevent or mitigate sagging, for instance, sagging that would occur due to the unusually high density of the fabric.

Articles

According to some embodiments, suitable articles can comprise one or more of garments, building materials, safety equipment, armor, body armor, protective apparel, marine repair materials, marine protective devices, boat hulls, vehicle chassis, or blast-resistant materials. Suitable garments can include one or more of shorts, compression shorts, running shorts, a shirt, a jacket, pants, a tank top, or Capri pants.

According to some embodiments, articles can comprise one or more fabrics as set forth herein, and may also include conventional textile fabrics. In embodiments comprising more than one kind of fabric, the fabrics may be layered, and/or arranged adjacent to one another in the manner of panels. For instance, the sides of a pair of shorts may comprise high-density fabric, while the back and front may comprise conventional fabric or layered fabrics. In some embodiments the various fabrics may be sewn together, or joined by other suitable means, but in some embodiments, disparate fabrics may be woven together to form a single unitary article without the need for sewing.

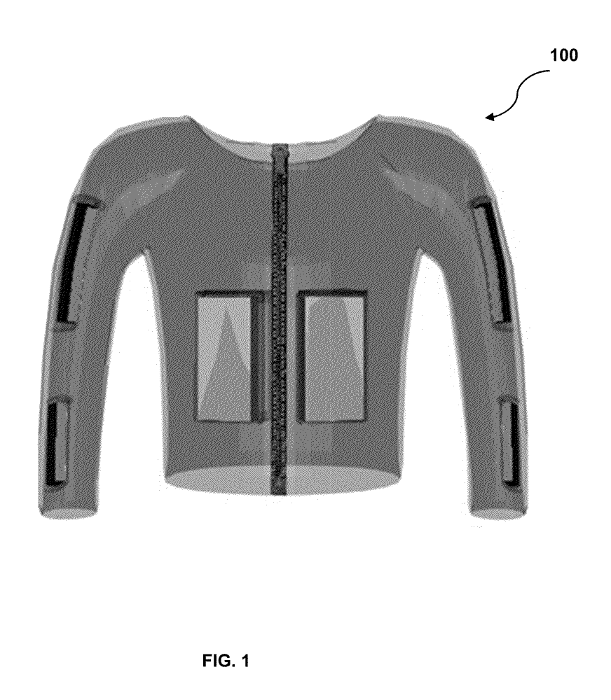

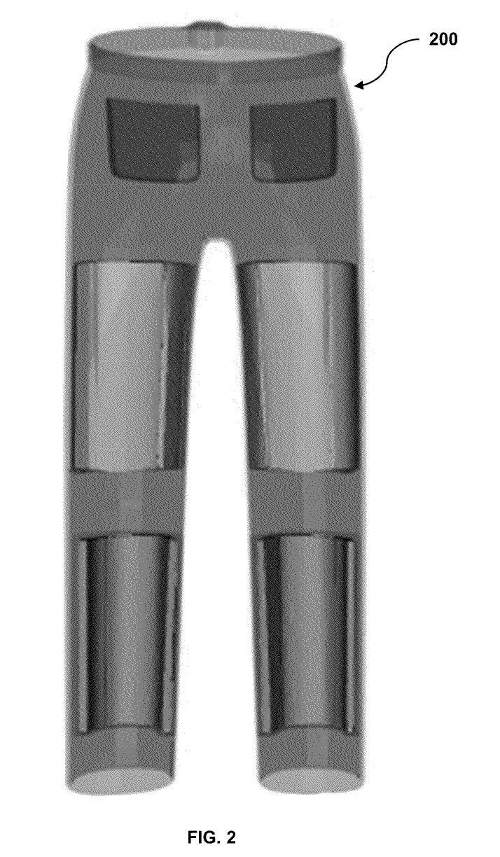

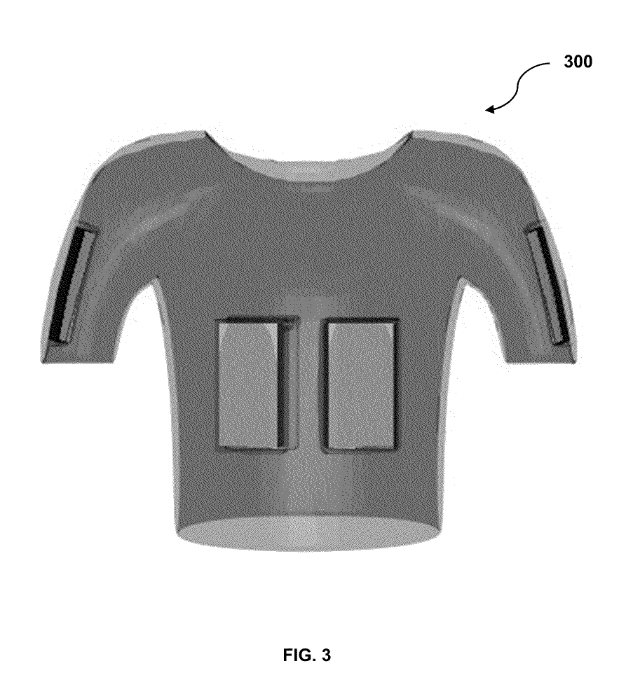

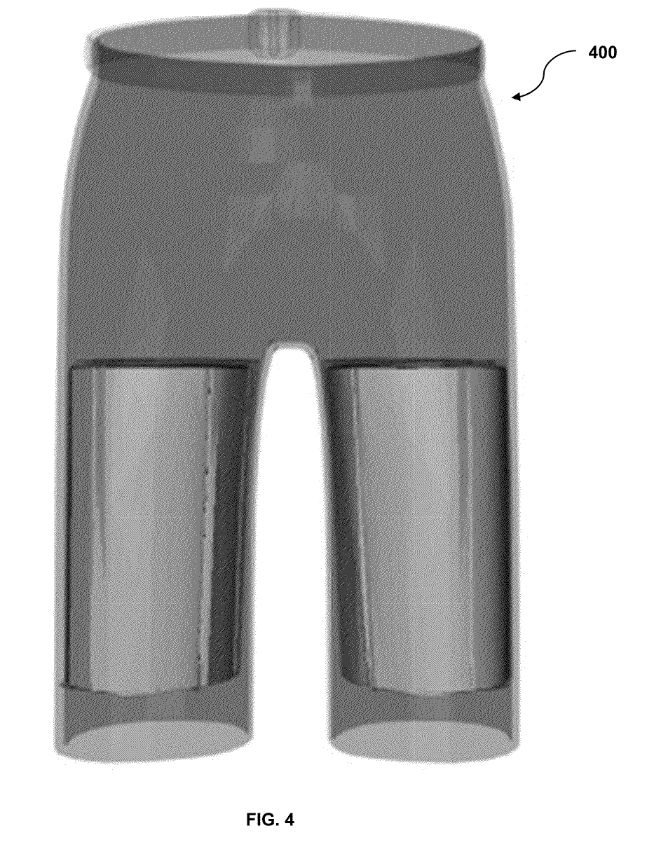

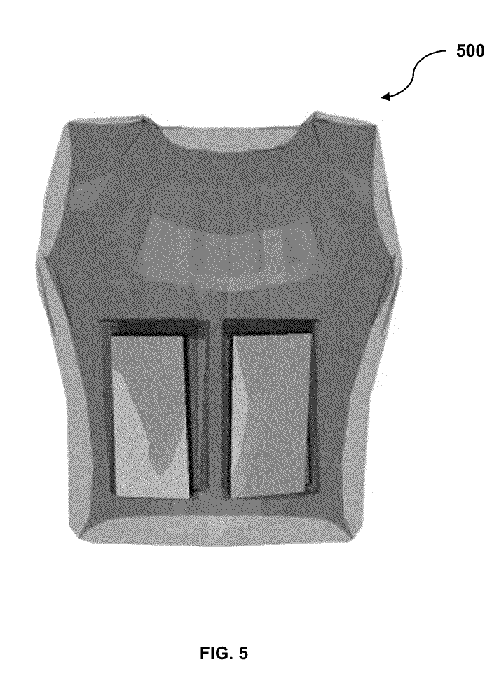

Turning to the drawings, FIG. 1 through 5 respectively illustrate embodiments comprising a jacket 100, a pair of pants 200, a shirt 300, a pair of shorts 400, and a vest 500. Each of these embodiments may be made from a fiber and/or fabric according to embodiments of the invention. For example, each embodiment 100, 200, 300, 400 and 500 may be made from one or more high-density textile fibers. Furthermore, the one or more high-density textile fibers may be formed into a high-density fabric which is used to construct the embodiments shown in FIG. 1 through 5. Still further, the high-density fibers may be blended with one or more ordinary textile fibers. As used herein, ordinary textile fibers include fibers that lack high-density elements such as, without limitation, metals, metal oxides, ceramics, and/or silicate glasses. Furthermore, ordinary textile fibers include those which have a density less than 1 g/mL.

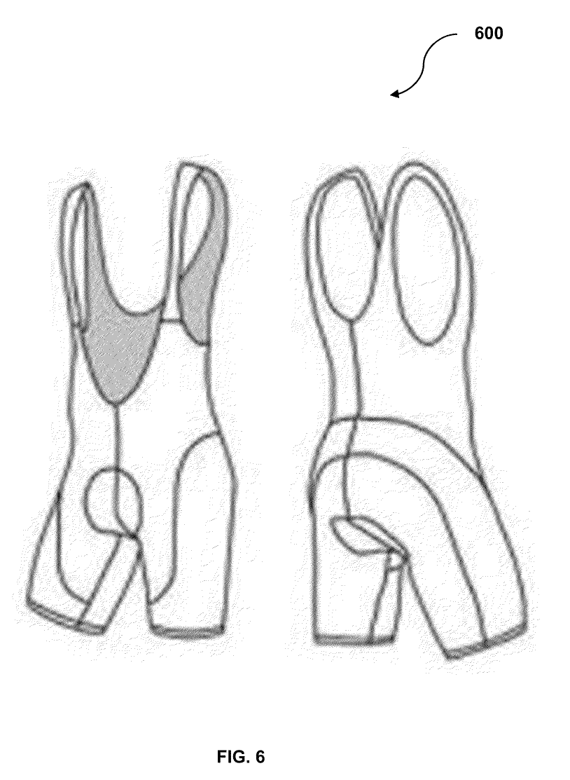

FIG. 6 is an opposing pair of perspective view drawings of an embodiment comprising a singlet 600, which is shown from both the back and front. This drawing shows a high-density garment embodiment 600 having a center of mass near the wearer's center of mass, and form-fitting to the wearer's body. Such an embodiment 600 may provide improved loading balance to the wearer while increasing the wearer's effective body mass. Thus, such an embodiment may be particularly beneficial to an individual engaged in exercise and may provide a superior physiological effect.

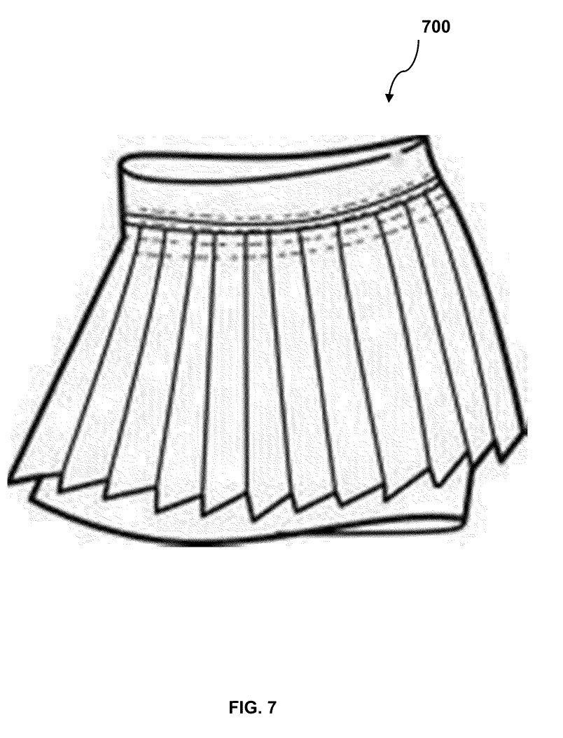

FIG. 7 is perspective view drawing of an embodiment comprising a miniskirt 700. According to this high-density garment embodiment 700, the center of mass of the article is close to that of the wearer, and thus may provide improved loading balance. Additionally, the embodiment 700 may be aesthetically pleasing and suitable for wearing either during exercise or during ordinary daily activities. Thus, a wearer can obtain a superior physiological benefit even when engaging in activities other than traditional exercise.

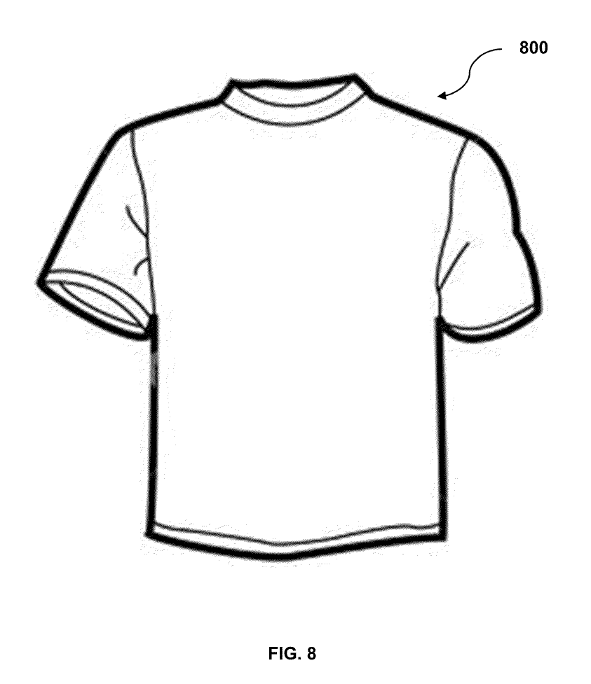

FIG. 8 is a perspective view drawing of an embodiment comprising a t-shirt 800. According to this high-density garment embodiment 800, the center of mass of the article is above that of the wearer and located roughly in the center chest area. Thus, such an embodiment 800 may be particularly beneficial to, for instance, a person engaged in abdominal or core-building exercises that require lifting the torso. Furthermore, the embodiment 800 can be worn under the individual's clothes. Therefore, the wearer can obtain a physiological benefit even when conducting activities outside of traditional exercise. For instance, such an embodiment 800 can be worn to the wearer's place of employment without being noticed or identifiable as an exercise device.

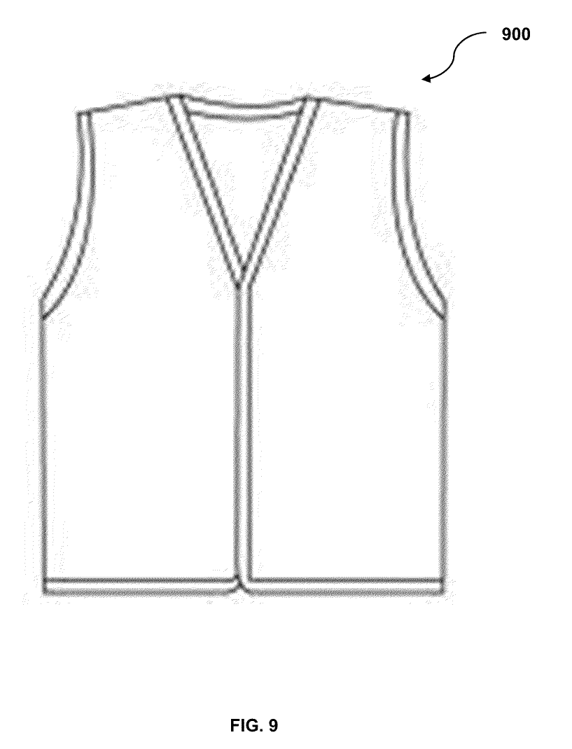

FIG. 9 is a front perspective view of a high-density garment embodiment comprising a vest 900. According to this high-density garment embodiment 900, the center of mass of the article is above that of the wearer and located roughly in the center chest area. Thus, such an embodiment 900 may be particularly beneficial to, for instance, a person engaged in abdominal or core-building exercises that require lifting the torso. In contrast to embodiment 800, the embodiment 900 shown in FIG. 9 can be worn over the clothing. Alternatively, the vest embodiment 900 can be worn instead of a shirt rather than over a shirt.

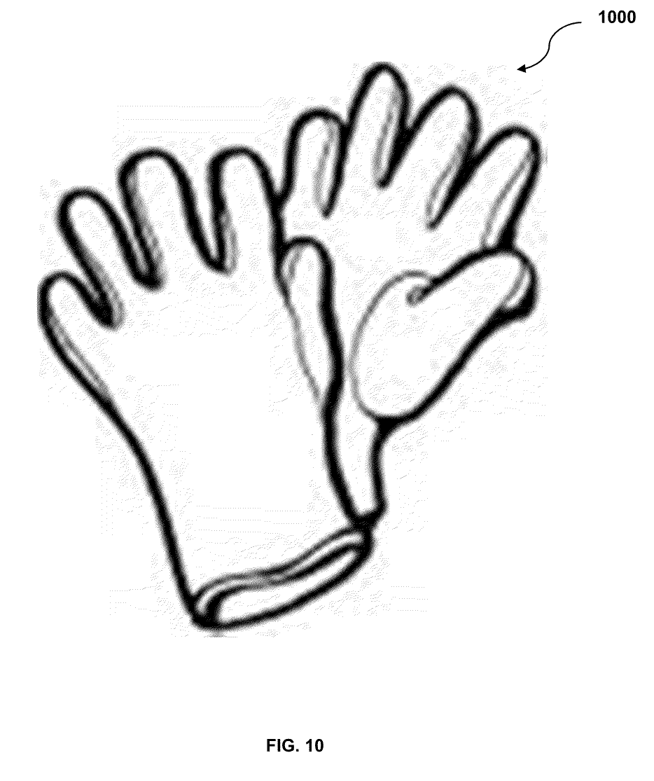

FIG. 10 is a perspective view drawing of a high-density garment embodiment comprising a pair of gloves 1000. According to this embodiment 1000 the center of mass of the article is located on the wearer's hands. Therefore, a wearer may obtain a physiological benefit when engaging in activities that use the hands. For instance, some forms of aerobic exercise involve hand motions and/or extension of the arm(s), which would be beneficially augmented by adding weight to the hands, while leaving the hands free. Furthermore, runners are known to hold their arms in a bent configuration while running rather than at their sides. Accordingly, runners may experience a superior physiological benefit by wearing the high-density glove embodiment 1000 shown in FIG. 10.

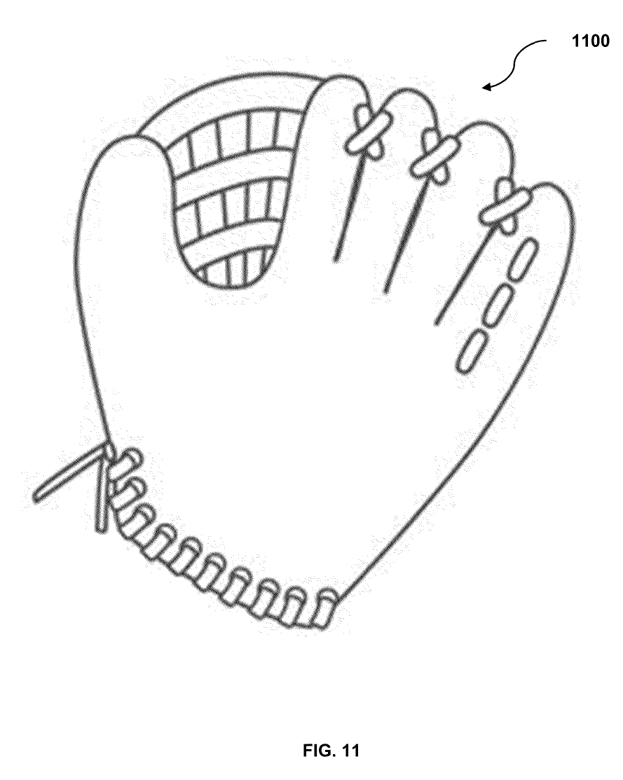

FIG. 11 is a front perspective view of a high-density sporting goods embodiment comprising a baseball mitt 1100. According to this embodiment 1100 the center of mass of the article is located on the wearer's hand. Therefore, a wearer may obtain a physiological benefit when engaging in sports training exercises that include using a baseball mitt. For example, a wearer may develop improved reaction time, motor control, strength and/or endurance as they relate to the act of catching baseballs. Accordingly, a wearer may wear the embodiment 1100 during training exercises and then wear a lighter ordinary baseball mitt at game time to reap the full benefit of his/her physiological improvement.

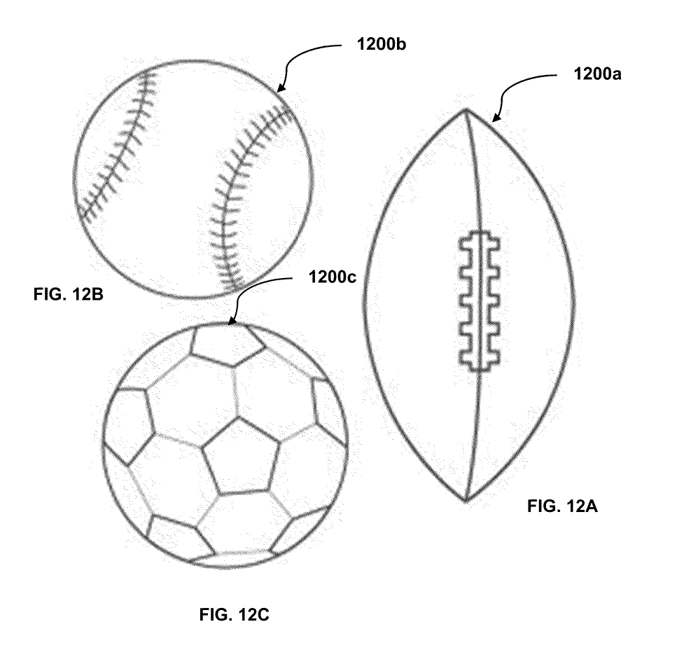

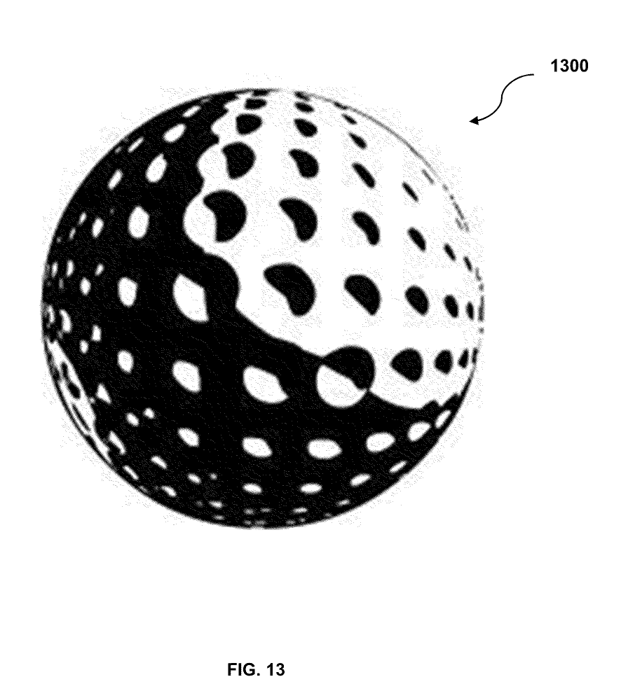

FIG. 12A-C and FIG. 13 are views of four game ball embodiments including a football 1200a, a baseball 1200b, a soccer ball 1200c, and a golf ball 1300. According to the illustrated embodiments 1200A-C and 1300, the game balls comprise a greater mass than ordinary game balls, and therefore require greater force to manipulate. Thus, embodiments according to FIG. 12A-C and FIG. 13 may confer a physiological benefit to players who practice with the high-density balls. Further, this benefit may result in improved play at game time.

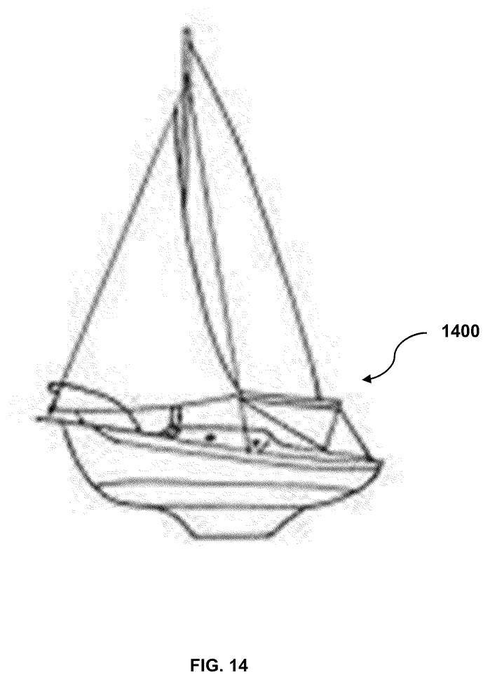

FIG. 14 is a perspective view of a boat having a hull 1400 made according to an embodiment of the invention. For example, a boat hull 1400 may comprise a high-density fiber according to an embodiment of the present invention, from which is made a woven structure. The woven structure may be impregnated with a polymer resin thus forming a composite structure suitable for constructing a boat hull. In some embodiments, the woven structure may be added to a mold and infused with molten resin, for instance, and then cured. The boat hull 1400 thus has a greater mass than ordinary boat hulls. Accordingly, a boat having such a hull has greater inertia, which may impart superior steering and/or handling characteristics during operation.

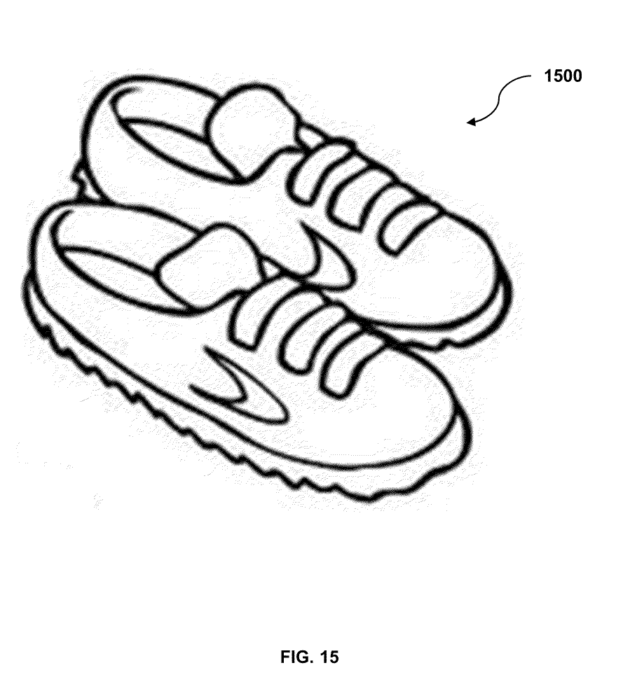

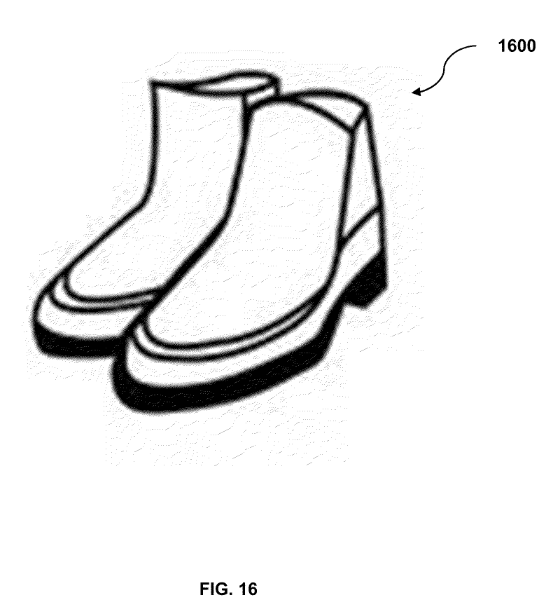

FIG. 15 and FIG. 16 are perspective views of embodiments comprising a pair of running shoes 1500 and a pair of boots 1600 respectively. The center of mass of each of these embodiments is located on the wearer's feet. Accordingly, the embodiments may confer a physiological benefit to the wearer through the act of walking or running by adding extra weight to the wearer's feet. Furthermore, the wearer may obtain such a benefit even when not necessarily engaged in traditional exercise activities because the embodiments may be suitable for everyday activities apart from exercise.

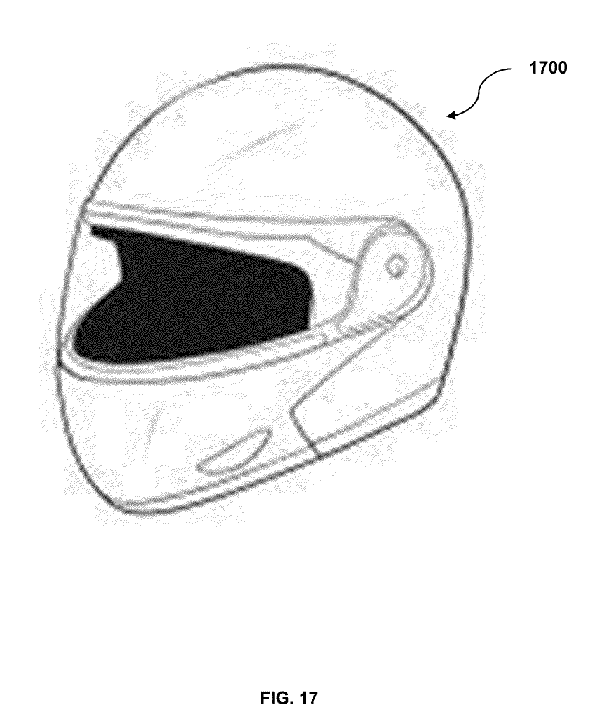

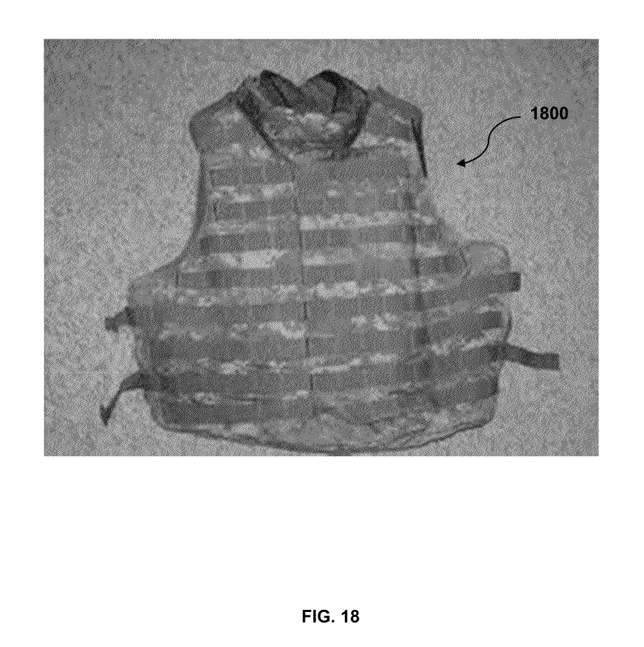

FIG. 17 and FIG. 18 illustrate protective garment embodiments comprising a helmet 1700 and a body armor vest 1800 respectively. According to these embodiments the wearer may obtain a protective benefit from the garment due to improved material characteristics including, for instance, impact resistance that may result from high-density fiber embodiments of the present invention and woven or nonwoven materials made therefrom.

Some embodiments having been described hereinabove, it will be apparent to those skilled in the art that the above methods and apparatuses may incorporate changes and modifications without departing from the general scope of this invention. It is intended to include all such modifications and alterations insofar as they come within the scope of the appended claims or the equivalents thereof.

I/We claim:

1. A textile fiber, comprising a density from about 1 to about 11 g/mL.

2. The textile fiber of claim 1 further comprising one or more of a glass fiber, a metal fiber, a conjugate fiber, a high-density polymer fiber, or a filled fiber.

3. The filled textile fiber of claim 2, wherein the fiber further comprises a matrix material selected from one or more of polyacetate, polyacrylate, polyacrylic, lyocell, modacrylic, nylon, polyamide nylon, polyphenol formaldehyde, polyvinyl alcohol, polyvinyl chloride, polyester, PET polyester, PBT polyester, acrylic polyesters, polyacrylonitrile, polyaramids, polyolefin, polypropylene, polyethylene, high performance polyethylene, ultra-high molecular weight polyethylene, polyurethane, polyurethane-polyurea copolymer, poly-1,3-propanediol, fiber glass, E-fiberglass, A-fiberglass, E-CR-fiberglass, C-fiberglass, D-fiberglass, R-fiberglass, or S-fiberglass, or any blend or copolymer thereof.

4. The filled textile fiber of claim 3, wherein the matrix material contains a filler material selected from one or more of metals, metal oxides, carbides, ceramics, or silicates.

5. The conjugate textile fiber of claim 2, wherein the fiber further comprises:
a core material selected from one or more of polyacetate, polyacrylate, polyacrylic, lyocell, modacrylic, nylon, polyamide nylon, polyphenol formaldehyde, polyvinyl alcohol, polyvinyl chloride, polyester, PET polyester, PBT polyester, acrylic polyesters, polyacrylonitrile, polyaramids, polyolefin, polypropylene, polyethylene, high performance polyethylene, ultra-high molecular weight polyethylene, polyurethane, polyurethane-polyurea copolymer, poly-1,3-propanediol, fiber glass, E-fiberglass, A-fiberglass, E-CR-fiberglass, C-fiberglass, D-fiberglass, R-fiberglass, or S-fiberglass, metals, copper, gold, silver, nickel, aluminum, iron, ferrous alloys, steel, spring steel, stainless steel or any blend or copolymer thereof; and
a sheath material selected from one or more of polyacetate, polyacrylate, polyacrylic, lyocell, modacrylic, nylon, polyamide nylon, polyphenol formaldehyde, polyvinyl alcohol, polyvinyl chloride, polyester, PET polyester, PBT polyester, acrylic polyesters, polyacrylonitrile, polyaramids, polyolefin, polypropylene, polyethylene, high performance polyethylene, ultra-high molecular weight polyethylene, polyurethane, polyurethane-polyurea copolymer, poly-1,3-propanediol, fiber glass, E-fiberglass, A-fiberglass, E-CR-fiberglass, C-fiberglass, D-fiberglass, R-fiberglass, or S-fiberglass, metals, copper, gold, silver, nickel, aluminum, iron, ferrous alloys, steel, spring steel, stainless steel, or any blend or copolymer thereof.

6. A textile yarn comprising at least a high-density textile fiber having a density from about 1 to about 11 g/mL.

7. The textile yarn of claim 6, wherein the high-density textile fiber comprises one or more of a glass fiber, a metal fiber, a conjugate fiber, a high-density polymer fiber, or a filled fiber.

8. The textile yarn of claim 7, wherein the filled fiber further comprises a matrix material selected from one or more of polyacetate, polyacrylate, polyacrylic, lyocell, modacrylic, nylon, polyamide nylon, polyphenol formaldehyde, polyvinyl alcohol, polyvinyl chloride, polyester, PET polyester, PBT polyester, acrylic polyesters, polyacrylonitrile, polyaramids, polyolefin, polypropylene, polyethylene, high performance polyethylene, ultra-high molecular weight polyethylene, polyurethane, polyurethane-polyurea copolymer, poly-1,3-propanediol, fiber glass, E-fiberglass, A-fiberglass, E-CR-fiberglass, C-fiberglass, D-fiberglass, R-fiberglass, or S-fiberglass, or any blend or copolymer thereof.

9. The textile yarn of claim 8, wherein the matrix material of the filled fiber contains a filler material selected from one or more of metals, metal oxides, carbides, ceramics, or silicates.

10. The textile yarn of claim 7, wherein the conjugate fiber further comprises:
a core material selected from one or more of polyacetate, polyacrylate, polyacrylic, lyocell, modacrylic, nylon, polyamide nylon, polyphenol formaldehyde, polyvinyl alcohol, polyvinyl chloride, polyester, PET polyester, PBT polyester, acrylic polyesters, polyacrylonitrile, polyaramids, polyolefin, polypropylene, polyethylene, high performance polyethylene, ultra-high molecular weight polyethylene, polyurethane, polyurethane-polyurea copolymer, poly-1,3-propanediol, fiber glass, E-fiberglass, A-fiberglass, E-CR-fiberglass, C-fiberglass, D-fiberglass, R-fiberglass, or S-fiberglass, metals, copper, gold, silver, nickel, aluminum, iron, ferrous alloys, steel, spring steel, or stainless steel, or any blend or copolymer thereof; and
a sheath material selected from one or more of polyacetate, polyacrylate, polyacrylic, lyocell, modacrylic, nylon, polyamide nylon, polyphenol formaldehyde, polyvinyl alcohol, polyvinyl chloride, polyester, PET polyester, PBT polyester, acrylic polyesters, polyacrylonitrile, polyaramids, polyolefin, polypropylene, polyethylene, high performance polyethylene, ultra-high molecular weight polyethylene, polyurethane, polyurethane-polyurea copolymer, poly-1,3-propanediol, fiber glass, E-fiberglass, A-fiberglass, E-CR-fiberglass, C-fiberglass, D-fiberglass, R-fiberglass, or S-fiberglass, metals, copper, gold, silver, nickel, aluminum, iron, ferrous alloys, steel, spring steel, or stainless steel, or any blend or copolymer thereof.

11. The textile yarn of claim 6 further comprising a textile fiber selected from one or more conventional textile fibers.

12. A textile fabric comprising one or more of a first yarn including a high-density fiber having a density from about 1 to about 11 g/mL.

13. The textile fabric of claim 12, wherein the first yarn comprises one or more of a glass fiber, a metal fiber, a conjugate fiber, a high density polymer fiber, or a filled fiber.

14. The textile fabric of claim 13 further comprising a woven reinforcing member adapted to support the weight of the fabric and mitigate or prevent the fabric from sagging under its own weight.

15. The textile fabric of claim 12, wherein the fabric weighs from about 1 lb/yd² to about 20 lb/yd².

16. An article including a fiber comprising a density from about 1 to about 11 g/mL.

17. The article of claim 16, further comprising a yarn having a high-density textile fiber comprising at least a density from about 1 to about 11 g/mL.

18. The article of claim 17 further comprising a fabric including one or more of a first yarn, the first yarn including a high-density fiber having a density from about 1 to about 11 g/mL.

19. The article of claim 16, wherein the article is selected from one or more of garments, building materials, safety equipment, armor, body armor, protective apparel, marine repair materials, marine protective devices, boat hulls, vehicle chassis, sporting goods, or blast-resistant materials.

20. The article of claim 19, wherein a garment can be selected from one or more of shorts, compression shorts, running shorts, a shirt, a jacket, a vest, a harness, pants, a tank top, a skirt, or Capri pants.
